(19) United States
(12) Patent Application Publication
(10) Pub. No.: US 2018/0342795 A1
(43) Pub. Date: Nov. 29, 2018

(54) MINIATURIZED ULTRA-WIDEBAND ANTENNA SYSTEM FOR MULTIPLE BIO-TELEMETRIC APPLICATIONS

(71) Applicant: UNIVERSITY OF ULSAN FOUNDATION FOR INDUSTRY COOPERATION, Ulsan (KR)

(72) Inventor: HYOUNG SUK YOO, Daegu (KR)

(21) Appl. No.: 15/640,237

(22) Filed: Jun. 30, 2017

(30) Foreign Application Priority Data
May 29, 2017 (KR) 1020170066061

(51) Int. Cl. H01Q 1/27; H01Q 1/48; H01Q 9/04; H01Q 1/38; A61B 5/07

(52) U.S. Cl. CPC H01Q 1/273; H01Q 1/48; A61B 5/076; H01Q 1/38; H01Q 9/045

(57) ABSTRACT

The present disclosure relates to an implantable communication device with an ultra-wideband antenna, and the implantable communication device according to an exemplary embodiment of the present disclosure includes: a sensor unit which obtains a biological signal in a body at a site where the implantable communication device is implanted; and an ultra-wideband antenna which transmits the obtained biological signal to the outside of the body. In this case, the ultra-wideband antenna includes: a ground patch which includes a first slot, a second slot, and a feed hole; a first dielectric layer which is formed on the ground patch; a radiation patch which is positioned on the first dielectric layer, and includes a third slot having a spiral shape, and a feed line; and a second dielectric layer which is formed on the radiation patch, the feed line is connected to a lower surface of the radiation patch, and vertically penetrates the feed hole, one end of the first slot and one end of the second slot are connected to each other, and a shape of the second slot depends on a shape of the third slot.

### CROSS-REFERENCE TO RELATED APPLICATIONS

This application claims the priority of Korean Patent Application No. 10-2017-0066061 filed on May 29, 2017, in the Korean Intellectual Property Office, the disclosure of which is incorporated herein by reference.

### BACKGROUND

### Field

The present disclosure relates to an implantable communication device with an ultra-wideband antenna.

### Description of the Related Art

With the introduction of micro-technologies and nano-technologies, researches are being actively conducted on medical implant technologies. For example, a capsule endoscope, an implant antenna, and the like have been devised to monitor a heart rate, a blood glucose level, a body temperature, and intracranial pressure.

In particular, the implant antenna is a medium for transmitting and receiving electromagnetic waves, and the implant antenna is one of the most important constituent elements of medical implant devices for monitoring an interior of the body. However, because the implant antenna needs to be inserted into the body, the implant antenna needs to be small in size, and it is difficult to implement a desired bandwidth, a transmission rate, or the like.

Thus, there is a need for the developments of an implant antenna and an implant communication device which support a wide bandwidth and are compact in size.

In this regard, Korean Patent No. 10-0878719 (entitled “Human Body Communication Method, Human Body Communication System, and Capsule Endoscope used for the same) discloses a human body communication method including steps of generating a potential difference between two transmitting electrodes installed on a surface of the capsule endoscope, supplying electric current from the transmitting electrode, which has a relatively higher electric potential between the two transmitting electrodes, to the interior of the human body so that the electric current flows along the surface of the human body, allowing the electric current, which flows along the surface of the human body, to induce voltage between two receiving electrodes installed on the surface of the human body, and allowing the electric current, which induces voltage between the two receiving electrodes, to flow back to the interior of the human body and to be sunk into the transmitting electrode which has a relatively lower electric potential between the two transmitting electrodes, in which a capsule endoscope, which is put into the human body, transmits signals to the outside of the human body.

### DOCUMENT OF RELATED ART

### Patent Document

(Patent Document 1) Korean Patent No. 10-0878719

### SUMMARY

The present disclosure has been made in an effort to solve the aforementioned problems in the related art, and an object of the present disclosure is to provide an implantable communication device with an ultra-wideband antenna.

Specifically, the present disclosure provides an implantable communication device which is applicable to various biometric fields and operates in the ISM band (902 to 908 MHz) which is a frequency band designated as a high-frequency energy source for industries in addition to wireless communication, that is, for scientific and medical purposes.

However, technical problems to be solved by the present exemplary embodiment are not limited to the aforementioned technical problem, and other technical problems may be present.

According to an aspect of the present disclosure, there is provided an implantable communication device including: a sensor unit which obtains a biological signal in a body at a site where the implantable communication device is implanted; and an ultra-wideband antenna which transmits the obtained biological signal to the outside of the body. In this case, the ultra-wideband antenna includes: a ground patch which includes a first slot, a second slot, and a feed hole; a first dielectric layer which is formed on the ground patch; a radiation patch which is positioned on the first dielectric layer, and includes a third slot having a spiral shape, and a feed line; and a second dielectric layer which is formed on the radiation patch, the feed line is connected to a lower surface of the radiation patch, and vertically penetrates the feed hole, one end of the first slot and one end of the second slot are connected to each other, and a shape of the second slot depends on a shape of the third slot.

According to any one of the aforementioned technical solutions, the exemplary embodiment of the present disclosure provides the implantable communication device with the ultra-wideband antenna.

Specifically, the implantable communication device with the ultra-wideband antenna according to the exemplary embodiment of the present disclosure operates in the ISM band (902 to 908 MHz) which is the frequency band designated as a high-frequency energy source for industries in addition to wireless communication, that is, for scientific and medical purposes. Therefore, the implantable communication device with the ultra-wideband antenna is applicable to various biometrics fields.

Meanwhile, the effects obtained by the present disclosure are not limited to the aforementioned effects, and other effects, which are not mentioned above, will be clearly understood by those skilled in the art from the following description.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a view illustrating an implantable communication device with an ultra-wideband antenna according to an exemplary embodiment of the present disclosure;

FIG. 2 is a side view of a first exemplary embodiment of a capsule-type implantable communication device with an ultra-wideband antenna according to a first exemplary embodiment of the present disclosure;

FIG. 3 is a front view of the implantable communication device of FIG. 2;

FIG. 4 is a rear surface view of the implantable communication device of FIG. 2;

FIG. 5 is a rear view of the implantable communication device of FIG. 2;

FIG. 6 is a bottom plan view of the implantable communication device of FIG. 2;

FIG. 7 is a detailed configuration view of the implantable communication device with the ultra-wideband antenna according to the first exemplary embodiment of the present disclosure illustrated in FIG. 1;

FIG. 8 is a perspective view of a second exemplary embodiment of a flat-type implantable communication device with an ultra-wideband antenna according to a second exemplary embodiment of the present disclosure;

FIG. 9 is a rear view of the implantable communication device of FIG. 8;

FIG. 10 is a detailed configuration view of the implantable communication device of FIG. 8; FIG. 11 is a schematic view of the ultra-wideband antenna according to the first and second exemplary embodiments of the present disclosure;

FIG. 12 is a top plan view of the ultra-wideband antenna illustrated in FIG. 11;

FIG. 13 is a side view of the ultra-wideband antenna illustrated in FIG. 11;

FIG. 14 illustrates a view of a ground patch of the ultra-wideband antenna according to the first and second exemplary embodiments of the present disclosure;

FIG. 15 illustrates a view of the radiation patch of the ultra-wideband antenna of FIG. 14;

FIG. 16 illustrates bandwidths and gains with respect to heights of a first dielectric layer according to the first and second exemplary embodiments of the present disclosure;

FIG. 17 is a table illustrating parameters for link design for wireless communication by using the ultra-wideband antenna according to the first and second exemplary embodiments of the present disclosure;

FIG. 18 is a graph illustrating system margins with respect to distances from the implantable communication device with the ultra-wideband antenna according to the first and second exemplary embodiments of the present disclosure;

FIG. 19 is a graph illustrating experimental return losses which are calculated by an FDTD simulation of the ultra-wideband antenna manufactured in accordance with the first and second exemplary embodiments of the present disclosure, and return losses which are measured in a Duke model implemented similar to an environment in the body;

FIG. 20 is a graph illustrating return losses of capsule and flat type communication devices including the ultra-wideband antenna manufactured in accordance with the first and second exemplary embodiments of the present disclosure; and

FIG. 21 is a table for comparing antenna performance of the ultra-wideband antenna according to the first and second exemplary embodiments of the present disclosure with antenna performance of an implant antenna in the related art.

### DETAILED DESCRIPTION OF THE EMBODIMENT

Hereinafter, exemplary embodiments of the present disclosure will be described in detail with reference to the accompanying drawings so that those skilled in the art to which the present disclosure pertains may easily carry out the exemplary embodiments. However, the present disclosure may be implemented in various different ways, and is not limited to the exemplary embodiments described herein. Further, a part irrelevant to the description will be omitted to clearly describe the present disclosure.

Throughout this specification and the claims, when one constituent element is referred to as being “directly connected to” another constituent element, one constituent element can be directly connected to the other constituent element, and one constituent element can also be “electrically connected to” the other element with other elements therebetween. In addition, unless otherwise described, the word “comprise” and variations such as “comprises” or “comprising” will be understood to imply the inclusion of stated elements, not the exclusion of any other elements.

In the present specification, the term “unit” includes a unit implemented by hardware, a unit implemented by software, and a unit implemented by hardware and software. In addition, a single unit may be implemented by using two or more pieces of hardware, and two or more units may be implemented by using a single piece of hardware.

In the present specification, some operations or functions described as being performed by a terminal or a device may be performed by a server connected to the terminal or the device. Likewise, some operations and functions described as being performed by the server may also be performed by the terminal or the device connected to the server.

Hereinafter, the exemplary embodiments according to the present disclosure will be described in detail with reference to the accompanying drawings. The same or corresponding constituent elements are assigned with the same reference numerals regardless of reference numerals, and the repetitive description thereof will be omitted.

In addition, in the description of the present disclosure, the specific descriptions of publicly known related technologies will be omitted when it is determined that the specific descriptions may obscure the subject matter of the present disclosure. In addition, it should be noted that the accompanying drawings are provided only to allow those skilled in the art to easily understand the spirit of the present disclosure, and the spirit of the present disclosure is not limited by the accompanying drawings.

Hereinafter, in the detailed description of the present disclosure, an implantable communication device refers to an implantable communication device with an ultra-wideband antenna.

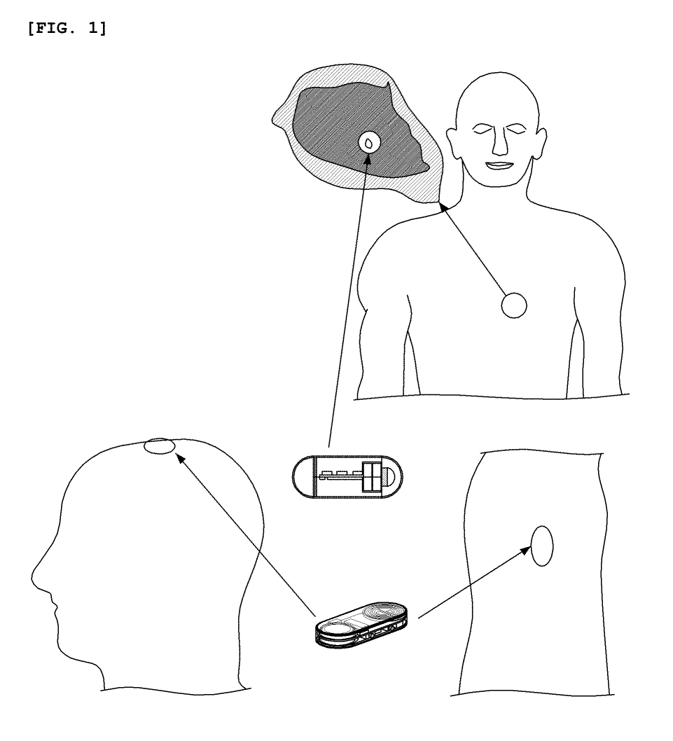

In addition, in FIGS. 1 to 15, a unit of numerical values is millimeter (mm), but the present disclosure is not limited thereto.

FIG. 1 is a view illustrating an implantable communication device with an ultra-wideband antenna according to an exemplary embodiment of the present disclosure.

FIGS. 2 to 6 are views illustrating an implantable communication device with an ultra-wideband antenna according to a first exemplary embodiment of the present disclosure.

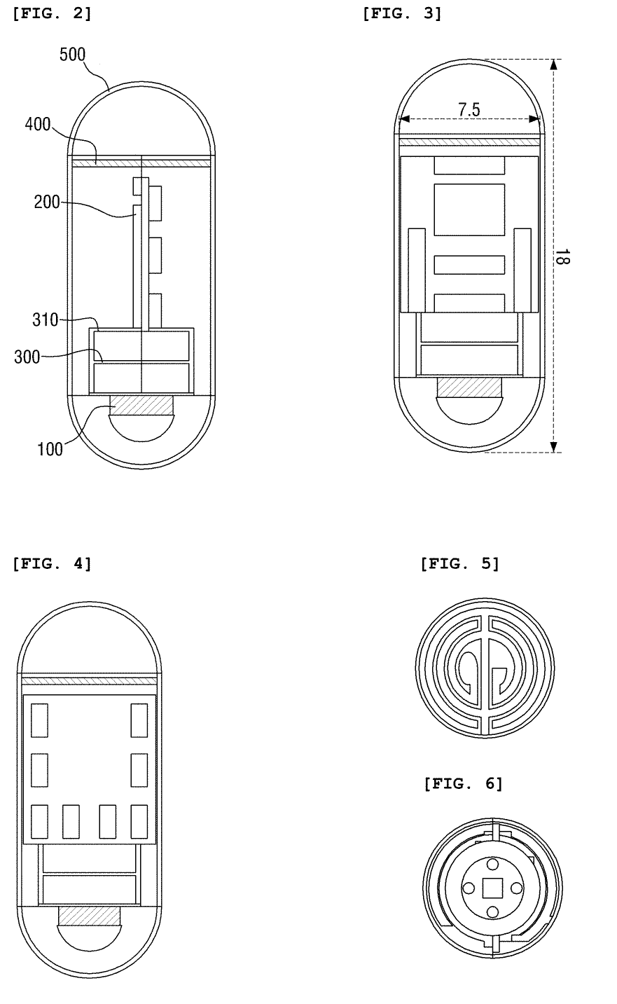

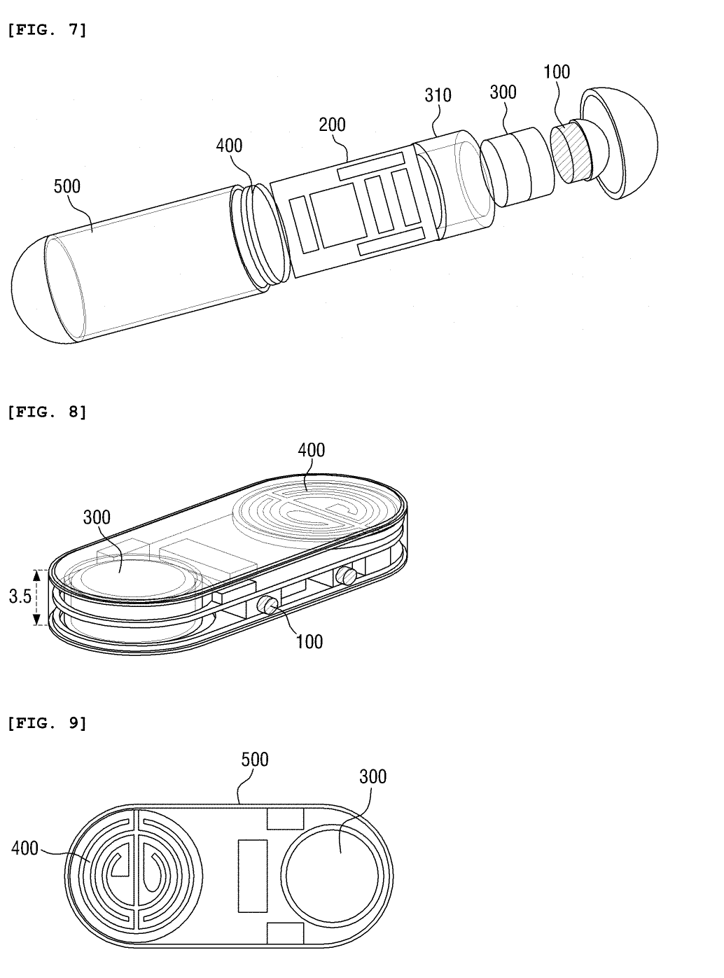

Specifically, FIG. 2 is a side view of a capsule type implantable communication device with an ultra-wideband antenna according to the first exemplary embodiment of the present disclosure, FIG. 3 is a front view, FIG. 4 is a rear surface, FIG. 5 is a rear view, and FIG. 6 is a bottom plan view.

FIG. 7 is a detailed configuration view of the implantable communication device with the ultra-wideband antenna according to the first exemplary embodiment of the present disclosure illustrated in FIG. 1.

In addition, FIGS. 8 to 10 are views illustrating an implantable communication device with an ultra-wideband antenna according to a second exemplary embodiment of the present disclosure.

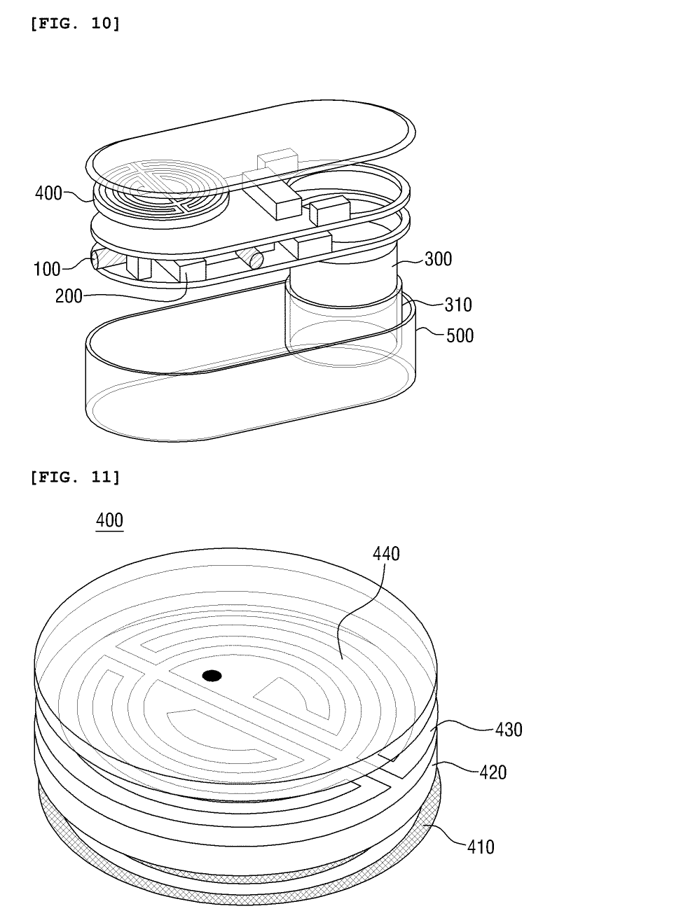

Specifically, FIG. 8 is a perspective view of a flat type implantable communication device, FIG. 9 is a rear view of the flat type implantable communication device, and FIG. 10 is a detailed configuration view of the flat type implantable communication device.

Referring to FIGS. 1 to 10, the implantable communication device with the ultra-wideband antenna according to the exemplary embodiment of the present disclosure may be implemented as a capsule type implantable communication device or a flat type implantable communication device as illustrated in FIGS. 1 to 10. Therefore, the implantable communication device according to the exemplary embodiment of the present disclosure is inserted into a skin, a cranium, and a heart, obtains state information about the skin, the cranium, and the heart, and transmits the state information to the outside.

Specifically, the capsule type implantable communication device illustrated in FIGS. 2 to 7 is applicable to tissue, such as the heart, located deep in the body, and the capsule type implantable communication device may be formed to have a length of about 18 mm and a diameter of about 7.5 mm.

In contrast, the flat type implantable communication device illustrated in FIGS. 8 to 10 is applicable to the skin, and the flat type implantable communication device may be formed to have a length of about 18 mm, a width of about 7.5 mm, and a height of about 3.5 mm.

In this case, as illustrated in FIGS. 1 to 7, both of the capsule and flat type implantable communication devices with an ultra-wideband antenna according to the exemplary embodiment of the present disclosure each include a sensor unit 100, a circuit unit 200, a power source unit 300, and an ultra-wideband antenna 400.

In this case, the respective constituent elements may be modularized by being accommodated in a single casing 500. Here, as an example, the casing 500 may have a thickness of 0.2 mm, and may be made of a biocompatible alumina ceramic material. In this case, permittivity (εr) of the alumina ceramic material may be about 9.8, and overall volume of the capsule and flat type implantable communication devices may be equal to or smaller than about 322.23 mm³ and 494.26 mm³, respectively, but the present disclosure is not limited thereto.

Hereinafter, the configurations of the capsule and flat type implantable communication devices according to the exemplary embodiment of the present disclosure will be described in more detail with respect to the drawings.

First, the sensor unit 100 may include various sensors in order to collect various types of information about an interior of a body of a human or an animal.

As an example, the sensor unit may include a sensor which collects any one of an image, pH, a temperature, pressure, and electrical impedance in the body.

Further, the circuit unit 200 may include an analog-digital converter ADC or a switch for processing information detected by one or more sensors. The circuit unit 200 may transmit the information, which is detected by the sensors of the ultra-wideband antenna 400, to a receiving unit (not illustrated) located out of the body.

Further, the power source unit 300 may include a portable battery. In this case, the portable battery may be fixed by a battery holder. In addition, the battery may be a small-sized battery that has a diameter of about 4.8 mm and a height of about 1.65 mm, but the present disclosure is not limited thereto.

The ultra-wideband antenna 400 radiates radio waves in the ISM band (902 to 908 MHz) which is a frequency band designated as a high-frequency energy source for industries in addition to wireless communication, that is, for scientific and medical purposes.

The ultra-wideband antenna 400 is integrated with the respective constituent elements that constitute the implantable communication device according to the exemplary embodiment of the present disclosure, and the ultra-wideband antenna 400 has appropriate gains and appropriate bandwidths so as to be smoothly operated at various sites in the body.

Hereinafter, the configuration of the ultra-wideband antenna 400 according to the exemplary embodiment of the present disclosure will be described in more detail with reference to the drawings.

FIG. 11 is a schematic view of the ultra-wideband antenna according to the first and second exemplary embodiments of the present disclosure.

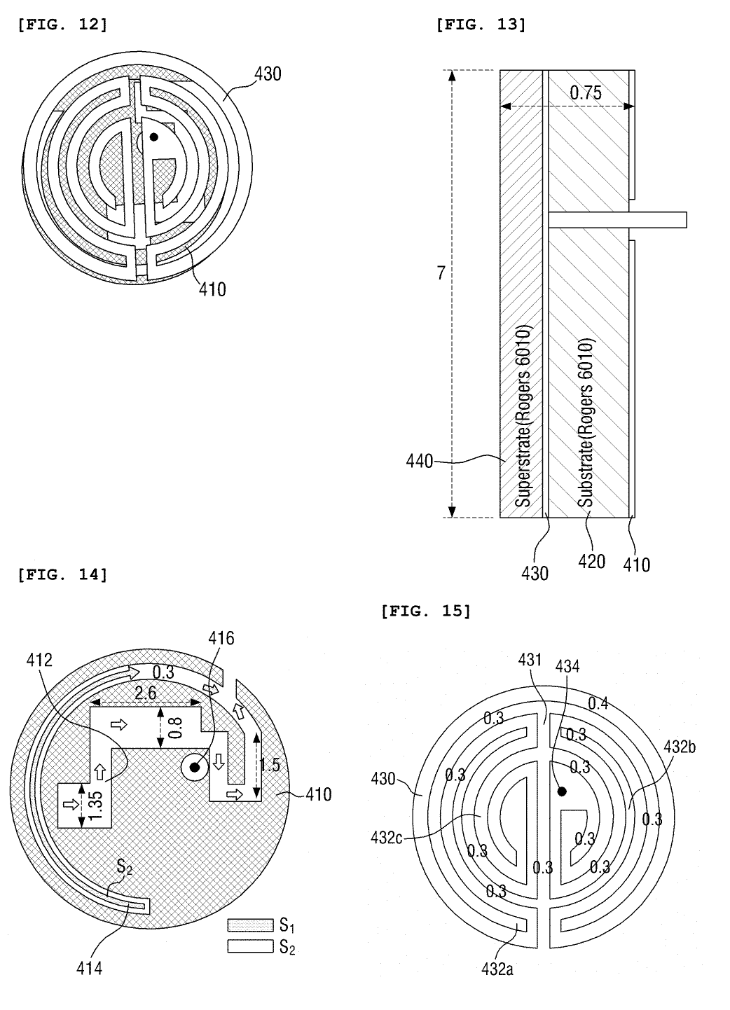

FIG. 12 is a top plan view of the ultra-wideband antenna illustrated in FIG. 11, and FIG. 13 is a side view of the ultra-wideband antenna illustrated in FIG. 11.

In addition, FIGS. 14 and 15 illustrate a ground patch and a radiation patch of the ultra-wideband antenna according to the first and second exemplary embodiments of the present disclosure. Specifically, FIG. 14 is a view illustrating the ground patch, and FIG. 15 is a view illustrating the radiation patch.

Referring to FIGS. 11,14 and 15, the ultra-wideband antenna 400 according to the first and second exemplary embodiments of the present disclosure may include a ground patch 410, a first dielectric layer 420 formed on the ground patch 410, a radiation patch 430 formed on the first dielectric layer 420, and a second dielectric layer 440 formed on the radiation patch 430. In this case, in the exemplary embodiment of the present disclosure, overall volume of the ultra-wideband antenna 400 may be about 28.85 mm³, but the present disclosure is not limited thereto.

First, as illustrated in FIG. 14, the ground patch 410 may include a first slot 412, a second slot 414, and an feed hole 416. Here, the first slot 412 and the second slot 414 may be formed by patterning an electric conductor to a preset pattern. In this case, one end of the first slot 412 and one end of the second slot 414 are connected to each other, and the connected ends of the first slot 412 and the second slot 414 may be formed in an open-ended shape so that the pattern is opened.

In addition, a shape of the first slot 412 may depend on a shape of a third slot formed in the radiation patch 430 which will be described below. According to the first and second exemplary embodiments of the present disclosure, the first slot 412 may improve system matching of the ultra-wideband antenna 400.

In addition, the second slot 414 has a larger area than the first slot 412, and the second slot 414 improves wideband characteristics of the antenna.

The first dielectric layer 420 may be made of a biocompatible dielectric material, and as an example, Biocompatible Rogers RO6010 (εr=10.2, loss tangent=0.0003) may be used, but the present disclosure is not limited thereto. In addition, according to the first and second exemplary embodiments of the present disclosure, a height of the first dielectric layer 420 may be calculated by a finite-difference time-domain (FDTD) simulation.

In this case, a thickness of the first dielectric layer 420 may be about 0.75 mm, but the present disclosure is not limited thereto.

Further, the radiation patch 430 may be formed on the first dielectric layer 420, and the radiation patch 430 is made of an electric conductive material.

In addition, the radiation patch 430 includes the third slot 432 and a feed line 434. In this case, the third slot 432 may have a radial (spiral) pattern. Specifically, the radial pattern of the third slot 432 may include an I-shaped slot which traverses a center of the radiation patch 430 from an outer circumferential surface of the radiation patch 430, and a plurality of C-shaped slots having a line width of about 0.3 mm. In this case, a length of the I-shaped slot 431 may be smaller by 0.7 mm than a diameter of the radiation patch.

Hereinafter, in the detailed description of the present disclosure, for ease of description, an opened portion of the C shape is defined as an end portion of the slot, and a length from the center of the radiation patch 430 to an outer circumferential surface of the end portion of the slot is defined as a radius of the slot.

Specifically, referring to FIG. 15, the radiation patch 430 may include a C-1 slot 432a which is spaced apart from the outer circumferential surface of the radiation patch 430 by about 0.4 mm and has a first radius and a line width of 0.3 mm, a C-2 slot 432b which has a second radius smaller by 0.6 mm than the first radius of the C-1 slot and has an end portion in a second direction opposite to (symmetrical by 180 degrees to) a first direction in which an end portion of the C-1 slot is positioned and formed, and a C-3 slot 432c which has a third radius smaller by 0.6 mm than the second radius of the C-2 slot and has an end portion formed in the first direction in which the end portion of the C-1 slot is positioned.

In other words, the plurality of C-shaped slots having the line width of 0.3 mm is formed in a direction toward the center of the radiation patch 430 from a position spaced 0.4 mm apart from the outer circumferential surface of the radiation patch 430, and the plurality of C-shaped slots may be formed to be spaced 0.3 mm apart from one another. As described above, the radial pattern of the third slot 432 extends an electrical length and improves a bandwidth (−10 dBi) of the antenna.

In addition, a frequency band radiated by the ultra-wideband antenna 400 according to the first and second exemplary embodiments of the present disclosure may be adjusted in accordance with the shape of the third slot 432 formed in the radiation patch 430.

In addition, one end of the feed line 434 is connected to a lower surface of the radiation patch 430, and the other end of the feed line 434 vertically passes through the feed hole 416 of the ground patch 410.

In this case, the feed line 434 may be used to supply electric power for radiating a frequency of the ultra-wideband antenna. In this case, the feed line 434 may have a radius of 0.3 mm, and the feed line 434 may be made of a conductive material having resistance of about 50Ω, but the present disclosure is not limited thereto.

Similar to the first dielectric layer 420, the second dielectric layer 440 may be made of a biocompatible dielectric material, and as an example, Biocompatible Rogers RO6010 (εr=10.2, loss tangent=0.0003) may be used.

Meanwhile, in the ultra-wideband antenna 400 according to the first and second exemplary embodiments of the present disclosure, widths, angles, intervals, positions, and the like of the first and second slots 412 and 414 of the ground patch 410 and the third slot 432 of the radiation patch 430 may be adjusted to control the bandwidth and the performance of the antenna.

In addition, the implantable communication device with the ultra-wideband antenna according to the first and second exemplary embodiments of the present disclosure may perform a parameter analysis in order to optimize a gain and achieve a bandwidth having small volume. Therefore, to perfectly perform the transmitting and receiving when performing wireless communication, respective parameter values may be controlled to be optimized so that the communication may be stably performed by predicting and calculating intensities, gains, noise factors, margins, and the like of respective elements at a transmitting end, a transmitting medium, and a receiving end.

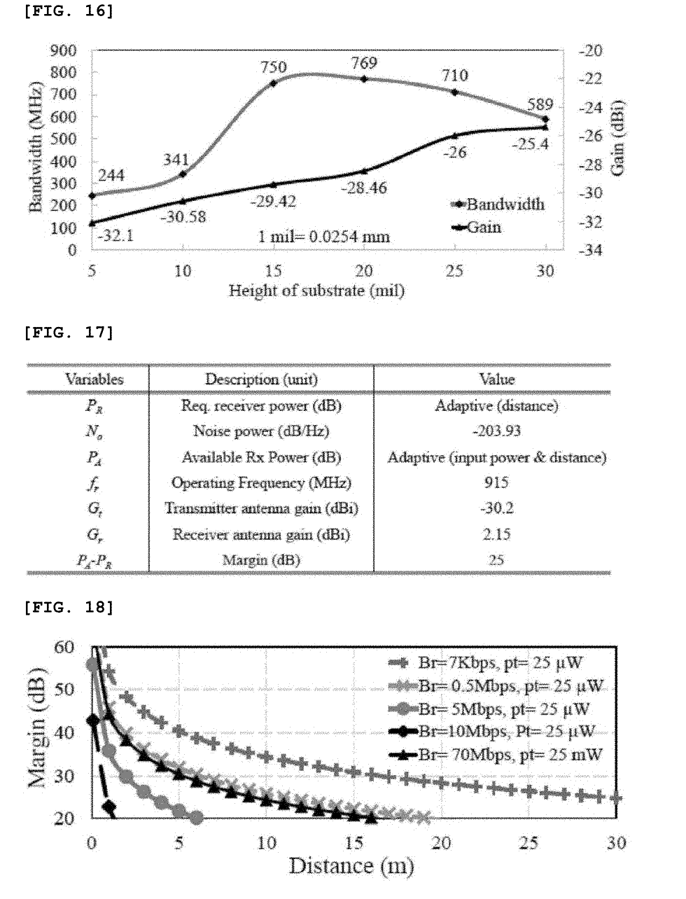

FIG. 16 illustrates bandwidths and gains with respect to heights of the first dielectric layer according to the first and second exemplary embodiments of the present disclosure.

Referring to FIG. 16, it can be seen that a trade-off relationship occurs between the gain and the bandwidth when a thickness of the first dielectric layer 420 is increased to be equal to or larger than a critical value. Therefore, the parameter analysis may be performed in order to optimize the gain and achieve the bandwidth having the small volume.

FIG. 17 is a table illustrating parameters for link design for wireless communication by using the ultra-wideband antenna according to the first and second exemplary embodiments of the present disclosure.

Specifically, the implantable communication device according to the first and second exemplary embodiments of the present disclosure performs wireless communication by using the ultra-wideband antenna 400 in order to perfectly perform the transmitting and receiving in respect to the link design, so that the communication may be stably performed by predicting and calculating intensities, gains, noise factors, margins, and the like of the respective elements at the transmitting end, the transmitting medium, and the receiving end.

FIG. 18 is a graph illustrating system margins with respect to distances from the implantable communication device with the ultra-wideband antenna according to the first and second exemplary embodiments of the present disclosure.

Referring to FIG. 18, input power Pt may depend on a data rate and a distance.

In addition, it was possible to obtain an experimental result which shows that a communication rate is decreased as a transmission rate is increased.

In addition, to verify performance of the implantable communication device with the ultra-wideband antenna according to the first and second exemplary embodiments of the present disclosure, return losses were measured in a Duke model similar to an environment in the body, and the return losses were compared with experimental data made through the FDTD simulation.

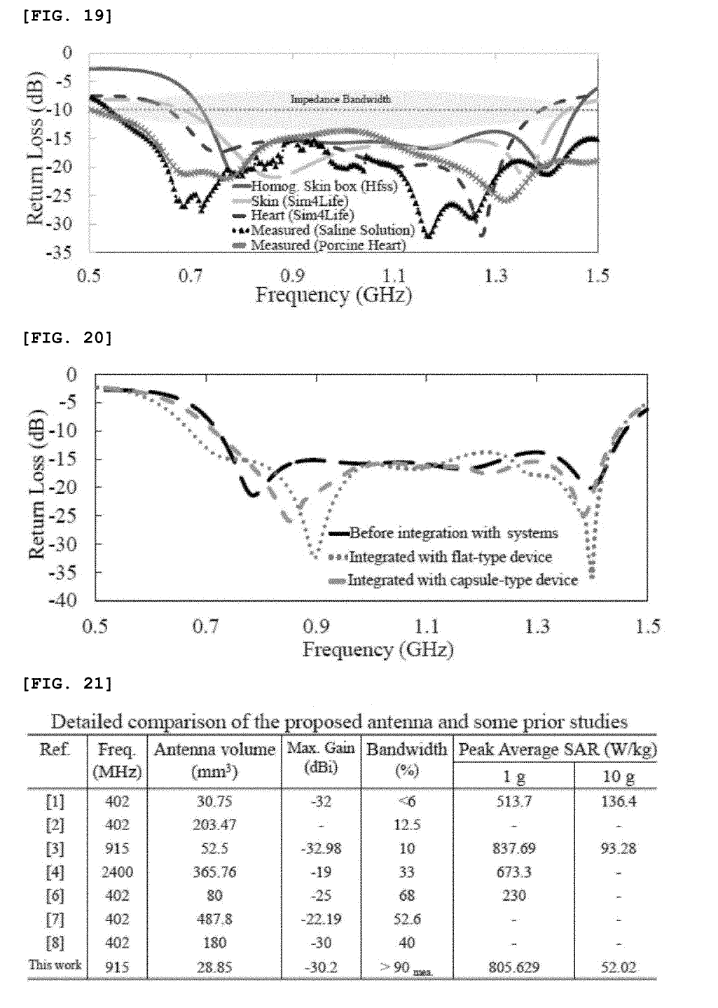

FIG. 19 is a graph illustrating experimental return losses which are calculated by the FDTD simulation of the ultra-wideband antenna manufactured in accordance with the first and second exemplary embodiments of the present disclosure, and the return losses which are measured in the Duke model implemented similar to an environment in the body.

In addition, FIG. 20 is a graph illustrating the return losses of the capsule and flat type communication devices including the ultra-wideband antenna manufactured in accordance with the first and second exemplary embodiments of the present disclosure.

Referring to FIGS. 19 and 20, an impedance bandwidth of the ultra-wideband antenna according to the first and second exemplary embodiments of the present disclosure was about 75% or higher in the experiment, and about 90% or higher when being measured in the Duke model.

Meanwhile, according to a result of performing a simulation after implementing an environment identical to the heart, the cranium, and the hand, peak gains at respective positions were −30.2 dBi, −27.7 dBi, and −28 dBi, respectively, and a peak value was decreased in accordance with a depth of an implant.

In addition, referring to FIG. 20, it can be seen from the experiment that good performance is still obtained when the ultra-wideband antenna according to the first and second exemplary embodiments of the present disclosure is coupled to respective constituent elements of a communication device, and it can be seen that a detuning phenomenon caused by the coupling is reduced.

FIG. 21 is a table for comparing antenna performance of the ultra-wideband antenna according to the first and second exemplary embodiments of the present disclosure with antenna performance of an implant antenna in the related art.

As illustrated in FIG. 21, a communication device including the ultra-wideband antenna provided by the present disclosure may be applied to various sites in the body, and the communication device is small in size and has resistance against the detuning.

Meanwhile, a peak SAR value is calculated based on the IEEE C 95.1-1999 and 95.1-2005 standard when the input power is 1 W, considering the concern about stability. In the case of the capsule type communication device which may be inserted into the heart, the peak SAR values were about 805.63 W/kg and about 52.02 W/kg based on 1 g and 10 g. That is, it can be seen that to maintain a stability limit value, maximum values of the input power of the communication device including the ultra-wideband antenna according to the exemplary embodiment of the present disclosure are 2.01 mW and 31.2 mW, respectively, based on 1 g and 10 g in accordance with the SAR standard, and thus the maximum values are the numerical values larger than 25 uW which is a maximum value of the permissible input power of the ultra-wideband antenna according to the exemplary embodiment of the present disclosure.

It will be appreciated that the exemplary embodiments of the present disclosure have been described above for purposes of illustration, and those skilled in the art may easily modify the present disclosure in other specific forms without changing the technical spirit or the essential features of the present disclosure. Therefore, it should be understood that the above-described exemplary embodiments are illustrative in all aspects and do not limit the present disclosure. For example, each component described to be of a single type may be implemented in a distributed manner. Likewise, components described to be distributed can be implemented in a combined manner.

The scope of the present disclosure is represented by the claims to be described below rather than the detailed description, and it should be interpreted that the meaning and scope of the claims and all the changes or modified forms derived from the equivalent concepts thereto fall within the scope of the present disclosure.

What is claimed is:

1. An implantable communication device with an ultra-wideband antenna, the implantable communication device comprising:
a sensor unit which obtains a biological signal in a body at a site where the implantable communication device is implanted; and
an ultra-wideband antenna which transmits the obtained biological signal to the outside of the body,
wherein the ultra-wideband antenna includes:
a ground patch which includes a first slot, a second slot, and a feed hole;
a first dielectric layer which is formed on the ground patch;
a radiation patch which is positioned on the first dielectric layer, and includes a third slot having a spiral shape, and a feed line; and
a second dielectric layer which is formed on the radiation patch,
the feed line is connected to a lower surface of the radiation patch, and vertically penetrates the feed hole,
one end of the first slot and one end of the second slot are connected to each other, and
a shape of the second slot depends on a shape of the third slot.

2. The implantable communication device according to claim 1, wherein the ultra-wideband antenna radiates radio waves of 902 MHz to 908 MHz.

3. The implantable communication device according to claim 2, wherein the first dielectric layer and the second dielectric layer are made of a biocompatible dielectric material.

4. The implantable communication device according to claim 3, wherein a thickness of the first dielectric layer and a thickness of the second dielectric layer are calculated by a finite-difference time-domain (FDTD) simulation.

5. The implantable communication device according to claim 2, wherein the third slot is formed in a radial (spiral) shape in order to improve an electrical length.

6. The implantable communication device according to claim 2, wherein performance of the antenna varies in accordance with at least one of a shape of the first slot, a shape of the second slot, and a shape of the third slot.

7. The implantable communication device according to claim 1, wherein the feed line electrically connects the ground patch and the radiation patch, and electric power is supplied to the ultra-wideband antenna through the feed line.
